(19) United States
(12) Patent Application Publication
(10) Pub. No.: US 2026/0216423 A1
(43) Pub. Date: Jul. 30, 2026

(54) CONCENTRIC TUBE FLUIDIC VALVE SYSTEM

(71) Applicants: Yue CHEN, Atlanta, GA (US); Nicholas BOULIS, Atlanta, GA (US); Anthony GUNDERMAN, Atlanta, GA (US); Anthony DONSANTE, Atlanta, GA (US); GEORGIA TECH RESEARCH CORPORATION, Atlanta, GA (US); EMORY UNIVERSITY, Atlanta, GA (US)

(72) Inventors: Yue CHEN, Atlanta, GA (US); Nicholas BOULIS, Atlanta, GA (US); Anthony GUNDERMAN, Atlanta, GA (US); Anthony DONSANTE, Atlanta, GA (US)

(21) Appl. No.: 19/147,493

(22) PCT Filed: Jan. 12, 2024
(86) PCT No.: PCT/US2024/011427

§ 371 Date: Jul. 11, 2025

Related U.S. Application Data
(60) Provisional application No. 63/479,765, filed on Jan. 13, 2023.

(51) Int. Cl. A61M 5/142; A61M 5/168

(52) U.S. Cl. CPC A61M 5/14276; A61M 5/16804; A61M 2005/14208; A61M 2205/0266; A61M 2230/62

(57) ABSTRACT

A system may include a pump. A system may include a catheter including a plurality of spaced apart orifices. A system may include a controller operatively coupled to the catheter, the controller being configured to control fluid flow to the catheter.

### CROSS-REFERENCE TO RELATED APPLICATIONS

This application claims the benefit of U.S. Provisional Patent Application No. 63/479,765 filed Jan. 13, 2023, the entire contents of which are incorporated herein by reference.

### BACKGROUND

Amyotrophic Lateral Sclerosis (ALS), also known as Lou Gehrig's disease, is a fatal neurological disorder characterized by motor neuron degeneration. The National ALS Registry identifies up to 31,000 persons currently suffering from ALS, with 6,700 new cases of ALS per year in the United States alone. Symptoms typically start with focal weakness in the limbs, but neuronal degeneration continues to propagate, causing muscle stiffness, spasticity, fasciculations, and nerve pain. As the disease progresses, patient independence is significantly reduced, affecting the quality of life of both patients and their caretaking family members. Ultimately, the disease exacerbates until diaphragmatic paralysis induces death, usually within 3 to 5 years. Despite its notoriety, ALS is an incurable disease. However, there are three FDA-approved treatment options: (i) riluzole, a glutamate blocker used to suppress excessive motor neuron firing, (ii) edaravone, a free radical scavenger that reduces oxidative stress to protect the degeneration of neuronal cells, and (iii) Relyvrio® (sodium phenylbutyrate and taurursodiol), which reduces neuronal death (although the mechanism of action is poorly understood). While these treatment modalities offer moderate improvements in disease progression and survival, they are currently only approved to be administered orally. As a consequence, the dosing of these drugs is limited by hepatic toxicity and asthenia.

### SUMMARY

In some aspects, the techniques described herein relate to a system including: a pump; a catheter including a plurality of spaced apart orifices; and a controller operatively coupled to the catheter, the controller being configured to control fluid flow to the catheter.

In some aspects, the techniques described herein relate to a system, further including a concentric tube valve (CTV) including an inner tube with a plugged distal end and the catheter having a plugged distal end.

In some aspects, the techniques described herein relate to a system, wherein the inner tube and the catheter include nitinol.

In some aspects, the techniques described herein relate to a system, wherein the inner tube includes a plurality of orifices.

In some aspects, the techniques described herein relate to a system, wherein the plurality of orifices of the inner tube are positioned to selectively allow flow through the plurality of orifices of the catheter.

In some aspects, the techniques described herein relate to a system, wherein the catheter includes a first orifice at a first position along a longitudinal axis of the CTV, and wherein the inner tube includes two orifices offset by 90 degrees and positioned at the first position along the longitudinal axis of the CTV and rotatable about the longitudinal axis.

In some aspects, the techniques described herein relate to a system, further including a concentric tube valve (CTV) including the catheter and configured as a valve.

In some aspects, the techniques described herein relate to a system, wherein the CTV includes a first set of orifices and a second set of orifices, and the CTV is configured as a three-position valve, wherein a first position is closed, a second position is open to a first set of orifices, and a third position is open to a second set of orifices, wherein the first set of orifices are optimized for a patient in an upright position, and wherein the second set of orifices are optimized for the patient in a supine position.

In some aspects, the techniques described herein relate to a system, further including an accelerometer operably coupled to the controller, wherein the controller includes a processor and a memory having instructions stored thereon, wherein execution of the instructions causes the processor estimate a pose of a patient and to control the pump based on the pose of the patient.

In some aspects, the techniques described herein relate to a system, wherein the pump is an implantable pump.

In some aspects, the techniques described herein relate to a system, wherein the implantable pump is configured to be implanted into an abdomen of a patient.

In some aspects, the techniques described herein relate to a system, wherein the controller is configured to receive instructions from a network connection.

In some aspects, the techniques described herein relate to a system, wherein the catheter defines a first aperture, a second aperture, and a third aperture spaced apart from one another along a longitudinal axis of the catheter.

In some aspects, the techniques described herein relate to a system, wherein the first aperture, the second aperture, and the third aperture are spaced equidistantly along the longitudinal axis.

In some aspects, the techniques described herein relate to a system, wherein the first aperture defines a first diameter, the second aperture defines a second diameter, and the third aperture defines a third diameter, wherein the third diameter is greater than the second diameter, and wherein the second diameter is greater than the first diameter.

In some aspects, the techniques described herein relate to a system, wherein the first aperture, the second aperture, and the third aperture are positioned adjacent a distal end of the catheter.

In some aspects, the techniques described herein relate to a system, wherein the catheter further defines a fourth aperture, a fifth aperture, a sixth aperture, a seventh aperture, and an eighth aperture, wherein the first aperture, the second aperture, the third aperture, the fourth aperture, the fifth aperture, the sixth aperture, the seventh aperture, and the eighth aperture are spaced equidistantly along the longitudinal axis, wherein diameters of the apertures increase from the first aperture positioned proximally, to the eight aperture positioned distally, and wherein the system is used to deliver a drug along a spine to treat Amyotrophic Lateral Sclerosis (ALS).

In some aspects, the techniques described herein relate to a computer implemented method of delivering a homogenous fluid, including: determining a plurality of orifice sizes to maintain homogenous flow rates through a catheter; and controlling a valve of the catheter so that fluid flows through orifices corresponding to the plurality of orifice sizes that will maintain the homogenous flow rates through the catheter.

In some aspects, the techniques described herein relate to a computer implemented method, further including controlling a plurality of valves of the catheter to select orifices of the catheter corresponding to the plurality of orifice sizes that will maintain homogeneous flow rates through the catheter.

In some aspects, the techniques described herein relate to a non-transitory computer readable medium having instructions stored thereon, wherein execution of the instructions by a processor causes the processor to: receive accelerometer information; determine a supine position or an upright position based on the accelerometer information; control operation of a pump based on the determination of the supine position or the upright position; and provide flow from the pump to a catheter defining a first aperture, a second aperture, and a third aperture, wherein the first aperture, the second aperture, and the third aperture are spaced equidistantly along a longitudinal axis, wherein the first aperture defines a first diameter, the second aperture defines a second diameter, and the third aperture defines a third diameter, wherein the third diameter is greater than the second diameter, and wherein the second diameter is greater than the first diameter.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF DRAWINGS

The device is explained in even greater detail in the following drawings. The drawings are merely exemplary and certain features may be used singularly or in combination with other features. The drawings are not necessarily drawn to scale.

FIG. 1 is a schematic representation of a system configuration of fluidic delivery system for administering a drug, according to some implementations.

FIG. 2A is a front view of a concentric tube valve (CTV) of the system configuration of FIG. 1 with a detailed view of the optimized proximal (e.g., 34.26 μm) and distal orifices (e.g., 311.00 μm), according to some implementations.

FIG. 2B is a front view of the CTV of FIG. 2A and illustrates a basic mode of operation. When a valve is closed, the flow is restricted, and when an inner tube is rotated and the valve is opened, flow occurs, according to some implementations.

FIG. 2C is a perspective view of the CTV of FIG. 2A and illustrates that a distal end of the CTV can be seen with the outer tube transparent in the right-hand image, according to some implementations.

FIG. 2D is a perspective view of the CTV of FIG. 2A and illustrates that, to open the valve, the inner tube is rotated 90° or 180° for an individual in an upright and supine position, respectively, according to some implementations.

FIG. 3 is a schematic representation of a spine and the system configuration of FIG. 1, according to some implementations.

FIG. 4A is a catheter of the system configuration of FIG. 1, according to some implementations.

FIG. 4B is another catheter of the system configuration of FIG. 1, according to some implementations.

FIG. 5 is a graph showing riluzole concentration vs. distance from a tip of the catheter of FIG. 4A, according to some implementations.

FIG. 6 is a flow chart of a method of delivering a homogenous fluid, according to some implementations.

FIG. 7 is a flow chart of another method of a method of delivering a homogenous fluid, according to some implementations.

### DETAILED DESCRIPTION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entirety and to the same extent as if each reference was individually incorporated by reference.

Amyotrophic Lateral Sclerosis (ALS) is a fatal neurological disorder characterized by motor neuron degeneration. Although there is no known cure, FDA-approved treatment options exist, such as riluzole and edaravone. However, these medications are limited to oral administration. Consequently, the dosing of these drugs is limited by hepatic toxicity and asthenia. Current intrathecal catheter designs distribute drugs heterogeneously. The present disclosure includes a fluidic system and model for homogenous drug delivery through a catheter.

In an example implementation, the present disclosure relates to a Concentric Tube Valve (CTV), and its corresponding envisioned system implementation for homogeneous intrathecal delivery of riluzole throughout the Cerebral Spinal Fluid (CSF) for patients suffering from ALS. The fluidic model is derived using the energy equation, and orifice diameters along the CTV are optimized to minimize fluidic head loss while maintaining a desired flow rate at each orifice.

An example implementation of the present disclosure device includes a concentric tube pair, where the inner tube and outer tube possess optimized orifice diameters along the length of the device that minimizes the fluidic head loss while maintaining a desired flow rate through each orifice. The orifices can be optimized using a derivation of the general energy equation for fluidic elements.

A physical prototype can be constructed and tested to analyze the flow rates out of the orifices of this prototype in different orientations and pressure gradients, ensuring model validation and homogeneous drug distribution. The methods and systems described herein can also investigate the diffusion process of the drug throughout the CSF in a bench-top setting using a custom-designed phantom with pressure gradients and flow profiles that emulate the physiological process of CSF biodistribution. Finally, as another non-limiting example, animal studies on dogs can be performed to evaluate the device's efficacy for long-term use.

Intrathecal drug delivery can be used as an alternative to oral or other forms of drug delivery. In intrathecal drug delivery, a drug is delivered directly into the cerebral spinal fluid (CSF) via a pump and a catheter. Intrathecal delivery is commonly used to bypass the blood-brain barrier for the administration of drugs to treat chronic cancer pain, spinal cord spasticity, and other central nervous systems (CNS) ailments. Advantages of this mode of delivery include: (i) circumvention of the blood-brain barrier (ii) systemic side-effect mitigation, (iii) reduced dosages (600 μg/day intrathecally vs. 60 mg/day orally in one instance), and (iv) continuous dosage regulation through pump administration. In recent work, Guttierez et al. and Boulis et al. have implemented intrathecal administration of riluzole in Göttingen minipigs and purpose-bred hound dogs, respectively, using a programmable infusion pump (SynchroMed II) and intrathecal catheter (Ascenda). In these studies, they were able to obtain higher spinal cord (SC) concentrations of riluzole above those achievable with oral therapy while eliminating the risk for adverse events associated with systemic drug exposure. However, current catheters used are designed to administer the drug at the distal end, relying on natural biodistribution to spread the drug homogeneously throughout the CSF. Designs typically consist of three different silicone-based styles: (i) open tip with single hole (Medtronic 8703), (ii) open tip with angled cut (Integra NL8507210), (iii) closed tip with rounded end (Spiegelberg Silverline), and (iv) closed-tip with distal side holes (Medtronic Ascenda 8780). Unfortunately, this method of distribution results in a heterogenous distribution within the CSF, with a 10-times greater amount of the drug being applied to neuronal cells near the location of the distal end of the catheter (near the cisterna magna). Further, these silicone devices are prone to fractures, perforations, and kinking.

The example implementation includes a system design and fluidic-modeling of a Concentric Tube Valve (CTV) for the homogeneous distribution of riluzole from the L2 vertebrae to the cisterna magna in both the upright and supine positions. As a non-limiting example, implementations of the present disclosure include a CTV, and a corresponding envisioned system implementation for homogeneous intrathecal delivery of riluzole throughout the CSF for patients suffering from ALS. The fluidic model is derived using the energy equation, and the orifice diameters along the CTV are optimized for given desired flow rates, known pressure distributions, and known design constraints (such as range of orifice diameter size), while minimizing system head-loss. The orifice diameters were optimized for a patient in both the upright and supine positions. It was observed that although there is a pressure gradient in the upright position, the energy needed to elevate the fluid along the length of the CTV increases proportionally with the decreasing pressure gradient. Thus, the optimized orifice sizes are the same for both the upright and supine positions.

The intrathecal drug delivery system disclosed herein aims to provide a homogeneous distribution of riluzole for ALS patients by controlling the flow rate along the intrathecal CTV. Although this example of the present disclosure relates to fluidic modeling for an intrathecal catheter in supine and upright positions, it should be understood that the present disclosure also can be used to perform homogenous flow rates across any catheter.

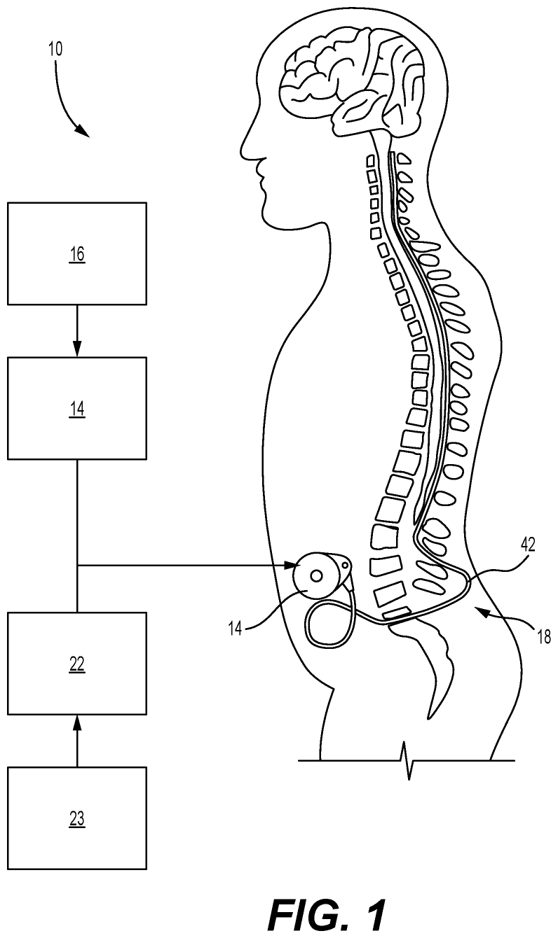

As shown in FIG. 1, a system 10 consists of three major components: (i) an implantable pump 14, (ii) a custom-designed CTV 18, and (iii) a controller 22 used to control the CTV 18. The pump 14 is a programmable, peristaltic, implantable pump (SynchroMed II 8637-40, Medtronic, Inc. USA). This pump 14 is commonly used for the intrathecal administration of opioids in CNS diseases, minimizing deviations from current intrathecal implantation clinical workflows. The pump 14 is located within the abdomen of a patient and is sutured to the fascia of the abdomen to ensure stability (see FIG. 1). The pump 14 is oriented so that the refill port rests against the abdominal wall, ensuring minimally invasive access via a syringe for pump refills. The flow rate of the pump 14 is regulated by the clinician through an application from a computing device 16 (e.g., a mobile device such as a table or a smartphone, a computer, a remotely located controller, a network, a cloud computing interface, etc.). This flow-rate is updated at regular clinical check-ups based on the patient's condition, ensuring optimal dosing. The CTV 18 is connected to the pump 14 using a luer lock mechanism and is routed into the subarachnoid space of the spinal cord at vertebrae L2 or lower using a 14-gauge Tuohy needle.

FIG. 1. illustrates the system configuration 10 of the intrathecal delivery system. The clinician provides a prescribed flow rate to the intrathecal pump while the CTV 18 ensures a homogeneous distribution of the drug riluzole throughout the cerebral spinal fluid.

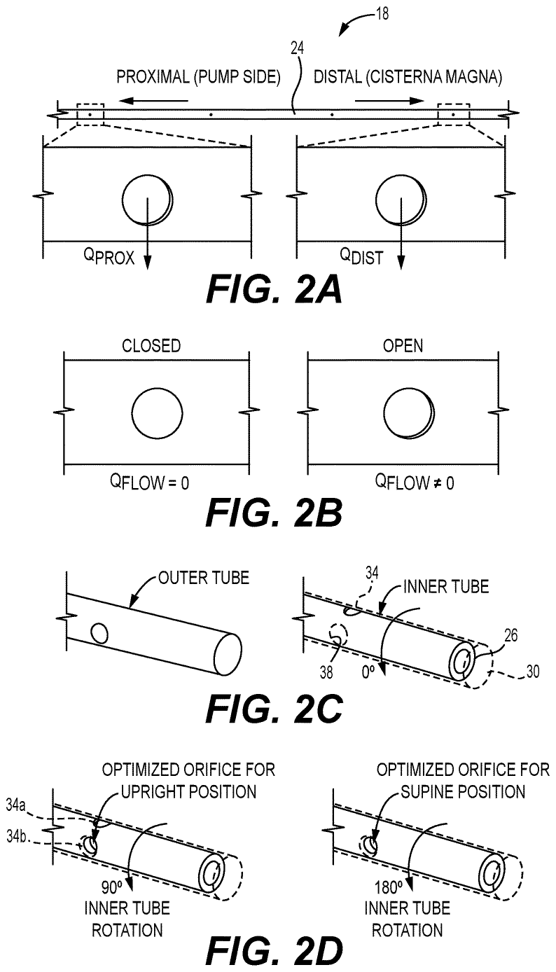

As shown in FIGS. 2A-D, the CTV 18 consists of a tube pair 24 including two nested, nitinol tubes, both with a plugged distal end. An inner tube 26 is a 24-gauge tube (OD: 0.566 mm, ID: 0.311 mm) and an outer tube 30 is a 20-gauge tube (OD: 0.908 mm, ID: 0.603 mm). The total length of the tube pair 24 was considered as 80 cm (typical for intrathecal catheters); however, the distance from the first orifice to the last orifice can be the average length of a human spinal cord (45 cm). The inner tube 26 includes inner orifices 34 and the outer tube 30 includes outer orifices 38, along the length of the tube pair 24 with varying diameters optimized to maintain a homogeneous flow rate throughout the CSF (FIG. 2A). In some implementations, and as shown in FIG. 2D, the outer tube 30 possesses one orifice 38 equidistant along the longitudinal axis of the CTV 18 starting at vertebrae L2, whereas the inner tube 26 possesses two orifices 34a and 34b offset by 90°. This orifice configuration enables the CTV 18 to act as a valve (see FIG. 2B) with three possible valve positions (i.e., a three-way valve). These positions include: (i) the valve 18 is closed (FIG. 2C), (ii) the valve 18 is opened to orifice set one (FIG. 2D), and (iii) the valve 18 is opened to orifice set two (FIG. 2D). The two sets of orifices 34 in the inner tube 26 enable the optimization of the CTV 18 for an individual in an upright position and in a supine position, where these positions result in differing pressure gradients in the spine due to gravity. Fabrication of these orifices 34, 38 would be performed using a femtosecond laser (Optec WS-Flex USP, Belgium), which can provide a kerf diameter of 22 μm (smallest feasible orifice size). The rotation angle of the inner tube 26 will be controlled by patient pose feedback provided to a controller through accelerometer pose estimation based on the outputs of an accelerometer 23.

FIGS. 2A-2D show one implementation of the CTV 18. (A) illustrates the CTV 18 with a detailed view of the optimized proximal (34.26 μm) and distal orifices (311.00 μm). The distal orifice diameter is larger than the proximal orifice diameter to maintain the desired flow rate through each orifice despite the significant head loss across the tube body. (B) Illustrates the basic mode of operation. When the valve 18 is closed, the flow is restricted. When the inner tube 26 is rotated and the valve 18 is opened, flow occurs. (C) Illustrates that the distal end of the CTV 18 can be seen with the outer tube 30 transparent in the right-hand image. (D) illustrates that, to open the valve 18, the inner tube 26 is rotated 90° or 180° for an individual in an upright and supine position, respectively.

The example system 10 described herein can be designed using methods based on energy equations.

Analyzing pressure loss across fluidic elements is derived by first starting with the energy equation of a fluidic system between two points of interest:

$${H}_{1} + \frac{m {u}_{1}^{2}}{2} + m g {z}_{1} + {Q}_{h e a t} = {H}_{2} + \frac{m {u}_{2}^{2}}{2} + m g {z}_{2} + {W}_{T} \qquad ( 1 )$$

where H is the enthalpy of the fluid, \vec{g} is the acceleration constant corresponding to gravity, z is the height of the fluid, Qₕₑₐₜ is additional heat added to the fluid, and W_{T} is the work done by the fluid on that element. Due to the low fluid speeds

$$( u < 100 \frac{m}{s} )$$

and the absence of added heat, the study can assume this is an adiabatic process. Thus, the change in enthalpy can be written as:

$${H}_{1} - {H}_{2} = - {\int }_{1}^{2} V d P \qquad ( 2 )$$

where V is the volume of the fluid and P is the pressure. Substituting 14 into 13 and dividing by the mass:

$$- {\int }_{1}^{2} \frac{1}{ρ} d P + \frac{{u}_{1}^{2}}{2} + g {z}_{1} = \frac{{u}_{2}^{2}}{2} + g {z}_{2} + {w}_{T} \qquad ( 3 )$$

where w_{T} is the work per unit mass. Evaluating the integral, dividing by gravity, and knowing

$$γ = ρ g and u = \frac{Q}{A} ,$$

the energy equation can be written in its general form for a pumping system as:

$$\frac{{P}_{1}}{γ} + \frac{{Q}_{1}^{2}}{2 {gA}_{1}^{2}} + {z}_{1} = \frac{{P}_{2}}{γ} + \frac{{Q}_{2}^{2}}{2 {gA}_{2}^{2}} + {z}_{2} + HL \qquad ( 4 )$$

where γ is the specific gravity of the fluid, Q is the volumetric flow-rate, A is the cross-sectional area, and HL is the head loss caused by the work done by the fluid, which can be written as:

$$HL = RQ \vert Q \vert \qquad ( 5 )$$

where R is the fluidic resistance.
In this work, R can be divided into two components. These components consist of the fluidic resistance due to friction and local resistance losses caused by inlets, outlets, and fittings. The fluidic resistance due to the fluidic friction R_{f} in the tube can be written as:

$${R}_{f} = ( \frac{8}{{gD}_{H}} ) \frac{fL}{{A}^{2}} \qquad ( 6 )$$

where f is the dimensionless friction factor with respect to flow, L is the length of the segment, and D_{H} is the hydraulic diameter. The friction factor is a highly non-linear function dependent on flow speed. However, Churchill developed an explicit equation for obtaining the friction factor based on the Reynold's number, hydraulic diameter, and material roughness (0.0001 mm in this work). The fluidic resistance due to the local resistance losses Rₗ can be written as:

$${R}_{l} = \frac{8}{{gA}^{2}} \sum K \qquad ( 7 )$$

where K is the dimensionless local resistance coefficient (from fittings, inlets, and outlets). In this work, local resistance losses take place in the form of the expansion of the fluid from the concentric tube valve into the spinal canal. The loss coefficient due to expansion can be written as:

$${K}_{expansion} = {( 1 - {( \frac{{D}_{1}}{{D}_{2}} )}^{2} )}^{2} \qquad ( 8 )$$

where D₂ is the diameter of the spinal canal (~12 mm) and D₁ is the orifice in the concentric tube valve that can be optimized to provide a desired flow-rate out of the orifice.

### Fluidic System Analysis and Catheter Optimization

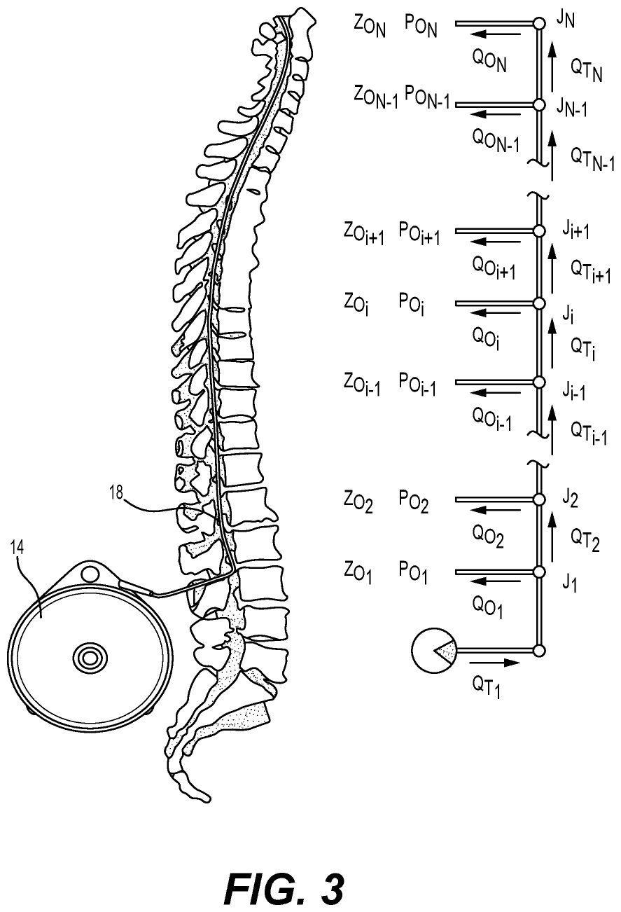

The concentric tube valve system is depicted in FIG. 3. The system consists of N orifices and N junctions Jᵢ in the spine corresponding to the locations of those orifices. Each orifice has a desired volumetric flow rate Qₒ_{ᵢ} defined by the clinician resulting in the following boundary condition.

$${Q}_{{T}_{1}} = {\sum }_{t = 1}^{N} {Q}_{{o}_{i}} \qquad ( 9 )$$

where Q_{T}_{₁} is the flow rate provided by the peristaltic pump through the first branch of the tube. To determine the orifice diameter dₒ_{ᵢ} at each junction, first write the energy equation from the junction Jᵢ to the node at orifice oᵢ in the spine can be written:

$${g ( {d}_{{o}_{i}} )}_{i} = \frac{{P}_{{o}_{i}}}{γ} + \frac{{Q}_{{o}_{i}}^{2}}{2 {gA}_{{o}_{i}}^{2}} + {z}_{{o}_{i}} + {HL}_{{J}_{i} \to {o}_{i}} = {H}_{{J}_{i}} \qquad ( 10 )$$

where Pₒ_{ᵢ}, Qₒ_{ᵢ}, Aₒ_{ᵢ}, and zₒ_{ᵢ} are the pressure, volumetric flow rate, cross-sectional area, and height of the fluid in the spine at the location in the spine corresponding to the position of the orifice i, respectively, H_{J}_{ᵢ} is the total head at the junction Jᵢ, and HL_{J}_{_{d i}}_{→o}_{ᵢ}; is the head loss across the branch between Jᵢ to the location in the spine corresponding to the position of orifice i, written as:

$${HL}_{{J}_{i} \to {o}_{i}} = {R}_{{l}_{{o}_{i}}} {Q}_{{o}_{i}} \vert {Q}_{{o}_{i}} \vert + {R}_{{f}_{{o}_{i}}} {Q}_{{o}_{i}} \vert {Q}_{{o}_{i}} \vert \qquad ( 11 )$$

where

$${R}_{{l}_{{o}_{i}}} and {R}_{{f}_{{o}_{i}}}$$

are the local resistance loss due to the expansion of the fluid and the frictional loss of the fluid, respectively, across the orifice to the spine. The energy equation from junction Jᵢ to junction Jᵢ₊₁ within the catheter can be written:

$$h ( {d}_{{o}_{i}} ) = {H}_{{J}_{i + 1}} + {HL}_{{J}_{i} \to {J}_{i + 1}} = {H}_{{J}_{i}} \qquad ( 12 )$$

where H_{J}_{ᵢ₊₁} is the total head at junction Jᵢ₊₁ and HL_{J}_{ᵢ}_{→J}_{ᵢ₊₁} is the head loss across the branch between Jᵢ to Jᵢ₊₁ written as:

$${HL}_{{J}_{i} \to {J}_{i + 1}} = {R}_{{f}_{{T}_{i + 1}}} {Q}_{{T}_{i + 1}} \vert {Q}_{{T}_{i + 1}} \vert \qquad ( 13 )$$

where

$${R}_{{f}_{{T}_{i + 1}}}$$

is the frictional loss of the fluid across the branch between junctions Jᵢ and Jᵢ₊₁ and Q_{T}_{ᵢ₊₁} is the volumetric flow-rate in the branch. Using the following equalities, the orifice diameter at each junction can be evaluated:

$$\begin{matrix}{f ( {d}_{{o}_{1}} )}_{1} = {g ( {d}_{{o}_{1}} )}_{1} - {h ( {d}_{{o}_{1}} )}_{1} = 0 \\ ⋮ \\ f {( {d}_{{o}_{i}} )}_{i} = {g ( {d}_{{o}_{i}} )}_{i} - {h ( {d}_{{o}_{i}} )}_{i} = 0 \\ ⋮ \\ f {( {d}_{{o}_{N}} )}_{N} = {g ( {d}_{{o}_{N}} )}_{N} - {H}_{{J}_{N}} = 0\end{matrix} \qquad ( 14 )$$

subject to the inequality constraint:

$${d}_{kerf} \leq {d}_{{o}_{i}} \leq {d}_{ID , CTV} \qquad ( 15 )$$

where d_{kerf} is the minimum diameter that can be cut by a femtosecond laser (22 μm) and d_{ID,CTV} is the inner diameter of the inner tube of the concentric tube valve. (14) provides infinitely many solutions. Thus, implementations of the present disclosure can minimize the amount of pump headed needed to overcome the associated head losses. In other words, for the parameter set dₒₚₜ=[dₒ_{₁} dₒ_{₂} . . . dₒ_{_{N}}]^{T}, the objective is to minimize the following:

$${d}_{opt} = arg {min}_{d} ( {\sum }_{i = 1}^{N} {HL}_{{J}_{i} \to {o}_{i}} + {\sum }_{i = 1}^{N - 1} {HL}_{{J}_{i} \to {J}_{i + 1}} ) \qquad ( 16 )$$

where dₒₚₜ is the optimal set of orifice diameters that minimize expended pump energy while maintaining the desired volumetric flow-rates.

FIG. 3 is a simplified view of the physical catheter-pump system 10 shown next to its corresponding fluidic energy schematic.

The CTV model was evaluated using the CTV 18 design with a total of N=10 orifices, resulting in an orifice spacing of 5 cm. The properties of riluzole used for fluidic analysis can be seen in Table I. A desired flow rate of 0.9 mL/day was used, and the desired flow-rates leaving each orifice were prescribed to be equal, i.e. Qₒ_{₁}=Qₒ_{ᵢ}=Qₒ_{₁₀}. The model was used to design the CTV 18 for both the supine and upright positions. In the supine position, the CSF pressure of 15 cm-H2O was used throughout (i.e., Pₒ_{ᵢ}=15 cm-H2O {i∈ [symbol] |1≤i≤10}. In the upright position, the pressure at the vertebrae L2 (i.e. Pₒ_{₁}) was considered to be 46.7 cm-H2O. Note that in the upright position the pressure head at the orifice will increase caudally along the spine due to the column of fluid and the acceleration of gravity, resulting in a pressure gradient. Using the energy equation, this gradient would imply that the pressure at the cisterna magna would be approximately 1.71 cm-H2O, which is supported by empirical data. Thus, using the energy equation, the pressure at each orifice can be calculated based on the pressure at the first orifice and the height at each following orifice.

TABLE 1
DRUG PROPERTIES

| Parameters | Units | Values |
|---|---|---|
| Drug | Name | Riluzole |
| Density | Kg/m³ | 1000 |
| Kinematic Viscosity | m²/s | 8.927E−07 |
| Specific Gravity | — | 1 |
| Flow-Rate | mL/day | 0.9 |

The results of the optimization process can be seen for the upright case and the supine case in Table 2 and Table 3, respectively. Provided in the table are the parameters that contribute to the energy equation based on the location in the spine and the corresponding optimized orifice diameters. Note that dₒ_{₁} and dₒ_{₁₀} correspond to the orifice diameters at vertebrae L2 and the cisterna magna, respectively.

The example implementations described herein, it was observed that the optimized orifice diameters are identical for both the upright case and the supine case. However, it should be understood that, in some implementations, the optimized orifice diameters can be different for the upright case and the supine case. This is explained using the energy equation. For example, consider the supine case. The both the pressure and the elevation remain constant throughout the spinal canal. Thus, the change in the left-hand side of (10) throughout the spinal is dependent only on the head loss. As a result, the only term contributing to orifice optimization is the head-loss term defined by (11). Conversely, in the upright case, although there exists a pressure gradient, the pressure at each orifice outlet decreases proportionally to the elevation of the fluid. Thus, the change in the two terms cancel, and the orifice optimization is again only based on the head-loss term defined by (11). Note that this phenomenon would not occur in systems with pressure gradients that are significantly influenced by factors independent of gravity, such as cardiac-based applications.

The optimized orifice diameters obtained do reflect results that would be expected. For example, consider a situation where all orifices are of the same dimension. The head-loss term has two components, head-loss due to friction and head-loss due to local resistances. If the orifices were all the same dimension, as the flow distances itself from the pump, frictional losses would normally reduce the head at each junction linearly, and the flow at orifice 10 (cisterna magna) would be significantly lower than the flow at orifice 1 (vertebrae L2). This would result in a heterogenous distribution of the drug. Conversely, as the tables suggest, by increasing the orifice size distally along the device (away from the pump), implementations of the present disclosure can maintain a desired head at each junction that ensures a consistent flow rate. Additionally, the objective of the optimization was to minimize expended effort by the pump, i.e., minimize head loss using (16). The orifice diameters in the tables support this. Note that head-loss decreases with increasing orifice diameter and that the final orifice is maximized, subject to the constraint (15). This implies that the head loss is minimized while maintaining the desired flow rates.

TABLE 2
UPRIGHT SYSTEM PARAMETERS

| Orifice | Pₒᵢ [cm H₂O] | Zₒᵢ [m] | Qₒᵢ [mL/day] | Dₒ [μm] |
|---|---|---|---|---|
| 1 | 46.70 | 0 | 0.09 | 33.38 |
| 2 | 41.70 | 0.05 | 0.09 | 35.29 |
| 3 | 36.70 | 0.10 | 0.09 | 37.57 |
| 4 | 31.71 | 0.15 | 0.09 | 40.38 |
| 5 | 26.21 | 0.20 | 0.09 | 43.92 |
| 6 | 21.71 | 0.25 | 0.09 | 48.60 |
| 7 | 16.71 | 0.30 | 0.09 | 55.22 |
| 8 | 11.71 | 0.35 | 0.09 | 65.65 |
| 9 | 6.71 | 0.40 | 0.09 | 86.32 |
| 10 | 1.71 | 0.45 | 0.09 | 311.00 |

TABLE 3
SUPINE SYSTEM PARAMETERS

| Orifice | Pₒᵢ [cm H₂O] | Zₒᵢ [m] | Qₒᵢ [mL/day] | Dₒ [μm] |
|---|---|---|---|---|
| 1 | 15.000 | 0.000 | 0.09 | 33.38 |
| 2 | 15.000 | 0.000 | 0.09 | 35.29 |
| 3 | 15.000 | 0.000 | 0.09 | 37.57 |
| 4 | 15.000 | 0.000 | 0.09 | 40.38 |
| 5 | 15.000 | 0.000 | 0.09 | 43.92 |
| 6 | 15.000 | 0.000 | 0.09 | 48.60 |
| 7 | 15.000 | 0.000 | 0.09 | 55.22 |
| 8 | 15.000 | 0.000 | 0.09 | 65.65 |
| 9 | 15.000 | 0.000 | 0.09 | 86.32 |
| 10 | 15.000 | 0.000 | 0.09 | 311.00 |

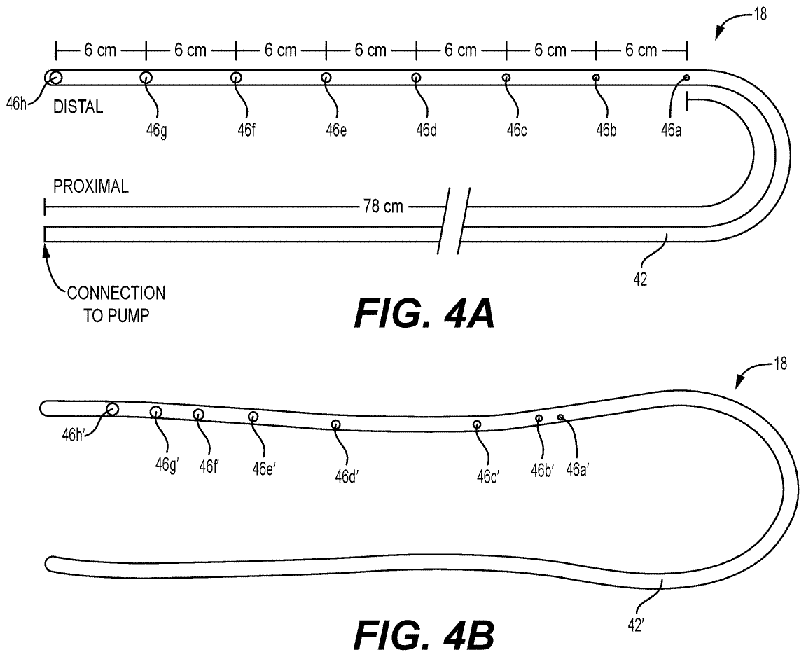

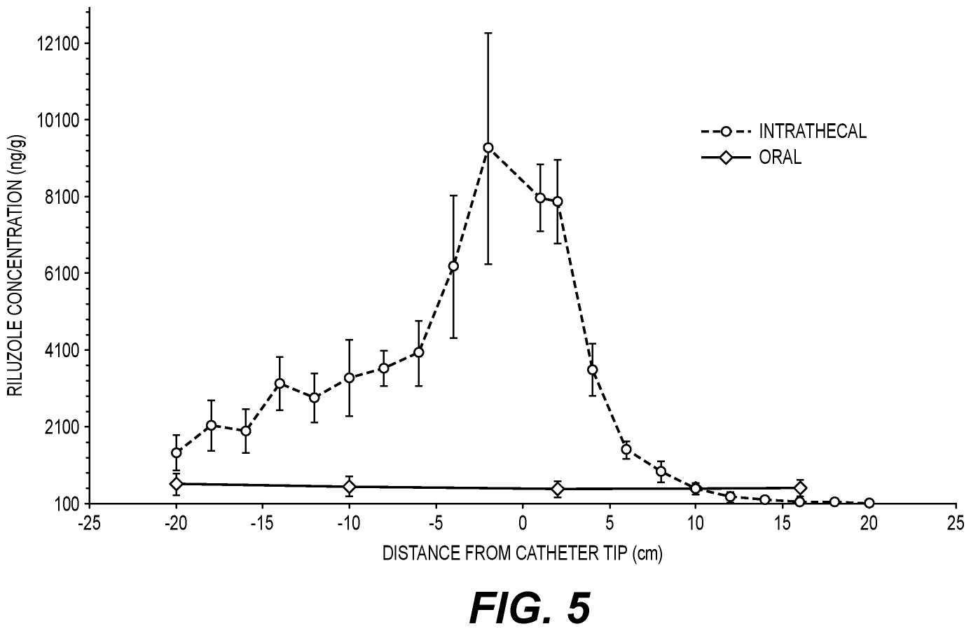

As shown in FIG. 4A, a catheter 42 (e.g., the tube pair 24) include eight apertures 46a-h. The human spinal cord is about 45 cm long. The tip can be placed at the top of the spinal cord to allow some delivery of the drug to the brain stem. The last orifice 46h is arranged about 3 cm from the end of the spinal cord. Based on the dog study, the peak drug spread can be about 10 cm from the end of the catheter 42 (as evidenced by the concentrations graphed in FIG. 5). In order to smooth out the ends of the distribution, the holes can be spaced every 6 cm to allow some overlap between each peak. The dimensions discussed herein are exemplary and the size, spacing, and number of the apertures 46a-h can be altered based on the calculations discussed above.

As shown in FIG. 4A, the catheter 42 defines a length of 78 cm of additional catheter to reach the pump 14. In other words, the catheter 42 is 120 cm in total. More or less catheter length can be provided. For example, the catheter may define a length of 80 cm to 150 cm. As shown in FIG. 1, when catheter 42 is implanted, the proximal end of the catheter 42 is tunneled under the skin from the patient's back to the front of the abdomen. The catheter 42 is looped to allow for some movement, and then any unneeded length is cut off before it is attached to the pump 14. So, ultimately, the length needs to be long enough to wrap halfway around the largest patient.

As shown in FIG. 4B, another CTV 18′ includes a catheter 42′ and apertures or orifices 46a′-h′ that are spaced apart from one another along a longitudinal axis of the catheter 42′. The orifices 46a-h shown in FIG. 4A are spaced equidistantly. The orifices 46a′-h′ are spaced non-uniformly. In some implementations, the spacing between orifices 46a′-h′ work in cooperation with the orifice diameters to provide homogeneous delivery of fluid along the length of the catheter 42′. In some implementations, the shapes of the orifices 46a-h or 46 a′-h′ are altered (e.g., star shaped, ovoid, etc.) to provide consistent and homogeneous flow.

In some implementations, the tube pair 24 includes the following parameters. The OD and ID are based on the valve type configuration (two tubes), as opposed to just a single tube with holes in it.
Inner Tube ID: 0.311 mm (24-gauge)
Inner Tube OD: 0.566 mm (24-gauge)
Outer Tube ID: 0.603 mm (20-gauge)
Outer Tube OD: 0.908 mm (20-gauge)
Orifice 1 Diameter: 36.0 um (micrometer) (Pump-side)
Orifice 2 Diameter: 39.0 um (micrometer)
Orifice 3 Diameter: 42.0 um (micrometer)
Orifice 4 Diameter: 46.0 um (micrometer)
Orifice 5 Diameter: 53.0 um (micrometer)
Orifice 6 Diameter: 63.0 um (micrometer)
Orifice 7 Diameter: 82.0 um (micrometer)
Orifice 8 Diameter: 311.0 um (micrometer) (Tip side)

Systems described herein can be used to treat tumors or other maladies where distributed drug delivery may be advantageous.

The systems described herein may be placed or positioned using multiple placement technologies (e.g., CTR, guidewire, tendon-based guidance, dexterous guidance system, etc.). In some implementations, the CTV discussed above does not include a valve but rather includes the catheter without a valve element or component. In some implementations, the CTV is actively adjusted based on activity (e.g., walking, sitting, running, etc.) or pressures (laying down vs. active or sitting, etc.). The system described above and in particular the catheter allows for a single placement, and then even delivery without movement of the catheter. This reduces required maintenance and increases the timing between required intervention by a clinician. In some implementations, the continuous administration and the direct administration eliminates the blood brain barrier that inhibits drug efficacy of orally taken drugs. In some implementations, the CTV includes components constructed of nitinol. In some implementations, other materials are used such as PEBAX. In some implementations, steerability allow the catheter to navigate the interfacial space from the cervical area upward to the base of the neck. In some implementations, a distal end of the catheter is coated in soft silicone to reduce damage or contusion during installation and placement. In some implementations, an ultrasound device is included in a tip of the catheter to aid in real time location tracking during placement (e.g., imaging, CAT, MRI, etc.) but imaging could also be integrated on the tip itself. In some implementations, a length of the catheter is sized to target a full length of the spine. In some implementations, at least 6 apertures are included. In some implementations, the largest aperture diameter is equal to the inner diameter of the outer tube and smallest aperture diameter is only limited by manufacturing capability.

While the discussion above is presented primarily with reference to riluzole and the treatment of ALS, it should be understood that the systems and structures discussed herein are only examples and concepts and structures presented in the claims can be applicable to other therapies, treatments, maladies, and/or delivery requirements. For example, catheters discussed herein may be implanted in a different portion of a patient's body (e.g., along a neck, along a bone of a leg or arm, etc.).

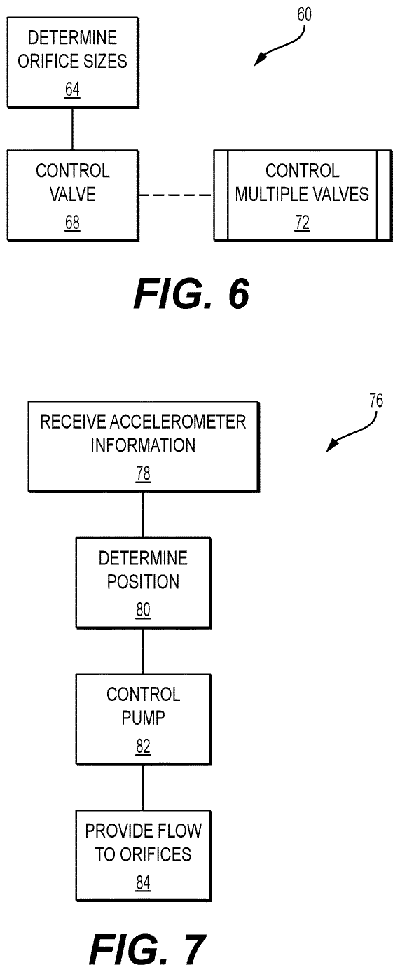

As shown in FIG. 6, a method 60 of delivering a homogenous fluid includes determining a plurality of orifice (e.g., the orifices 46a-h) sizes to maintain homogenous flow rates through a catheter (e.g., the catheter 42) at step 64, and controlling a valve (e.g., the CTV 18) of the catheter at step 68 so that fluid flows through orifices corresponding to the plurality of orifice sizes that will maintain the homogenous flow rates through the catheter. In some implementations, the method 60 also includes controlling a plurality of valves of the catheter at step 72 to select orifices of the catheter corresponding to the plurality of orifice sizes that will maintain homogeneous flow rates through the catheter.

As shown in FIG. 7, a method 76 includes receiving accelerometer information at step 78 (e.g., with the accelerometer 23), determining a supine position or an upright position based on the accelerometer information at step 80 (e.g., with the controller 22), controlling operation of a pump (e.g., the pump 14) based on the determination of the supine position or the upright position at step 82, and providing flow from the pump to a catheter (e.g., the catheter 42) defining a first aperture (e.g., orifice 46a), a second aperture (e.g., orifice 46c), and a third aperture (e.g., orifice 46f) at step 84. The first aperture, the second aperture, and the third aperture are spaced equidistantly along a longitudinal axis. The first aperture defines a first diameter, the second aperture defines a second diameter, and the third aperture defines a third diameter. The third diameter is greater than the second diameter. The second diameter is greater than the first diameter. In some implementations, a non-transitory computer readable medium having instructions stored thereon is provided, wherein execution of the instructions by a processor causes the processor to implement the method 76.

Example Computing System. The exemplary system and method may be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an implementation, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an implementation, virtualization software may be employed by the computing device to provide the functionality of a number of servers that are not directly bound to the number of computers in the computing device. For example, virtualization software may provide twenty virtual servers on four physical computers. In an implementation, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

Computing devices may have additional features/functionality. For example, the computing device may include additional storage such as removable storage and non-removable storage including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input device(s) such as keyboards, keypads, switches, dials, mice, trackballs, touch screens, voice recognizers, card readers, paper tape readers, or other well-known input devices. Output device(s) such as printers, video monitors, liquid crystal displays (LCDs), touch screen displays, displays, speakers, etc., may also be included. The additional devices may be connected to the bus in order to facilitate the communication of data among the components of the computing device. All these devices are well known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

It must also be noted that, as used in the specification and the appended claims, the singular forms “a,” “an,” and “the” include plural referents unless the context clearly dictates otherwise. Ranges may be expressed herein as from “about” or “approximately” one particular value and/or to “about” or “approximately” another particular value. When such a range is expressed, other exemplary implementations include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example implementations, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used.

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

1. A system comprising:
a pump;
a catheter including a plurality of spaced apart orifices; and
a controller operatively coupled to the catheter, the controller being configured to control fluid flow to the catheter.

2. The system of claim 1, further comprising a concentric tube valve (CTV) including an inner tube with a plugged distal end and the catheter having a plugged distal end.

3. The system of claim 2, wherein the inner tube and the catheter comprise nitinol.

4. The system of claim 2, wherein the inner tube comprises a plurality of orifices.

5. The system of claim 2, wherein the plurality of orifices of the inner tube are positioned to selectively allow flow through the plurality of orifices of the catheter.

6. The system of claim 2, wherein the catheter comprises a first orifice at a first position along a longitudinal axis of the CTV, and
wherein the inner tube comprises two orifices offset by 90 degrees and positioned at the first position along the longitudinal axis of the CTV and rotatable about the longitudinal axis.

7. The system of claim 1, further comprising a concentric tube valve (CTV) including the catheter and configured as a valve.

8. The system of claim 7, wherein the CTV comprises a first set of orifices and a second set of orifices, and the CTV is configured as a three-position valve,
wherein a first position is closed, a second position is open to a first set of orifices, and a third position is open to a second set of orifices,
wherein the first set of orifices are optimized for a patient in an upright position, and
wherein the second set of orifices are optimized for the patient in a supine position.

9. The system of claim 1, further comprising an accelerometer operably coupled to the controller,
wherein the controller includes a processor and a memory having instructions stored thereon, wherein
execution of the instructions causes the processor estimate a pose of a patient and to control the pump based on the pose of the patient.

10. The system of claim 1, wherein the pump is an implantable pump.

11. The system of claim 10, wherein the implantable pump is configured to be implanted into an abdomen of a patient.

12. The system of claim 1, wherein the controller is configured to receive instructions from a network connection.

13. The system of claim 1, wherein the catheter defines a first aperture, a second aperture, and a third aperture spaced apart from one another along a longitudinal axis of the catheter.

14. The system of claim 13, wherein the first aperture, the second aperture, and the third aperture are spaced equidistantly along the longitudinal axis.

15. The system of claim 13, wherein the first aperture defines a first diameter, the second aperture defines a second diameter, and the third aperture defines a third diameter,
wherein the third diameter is greater than the second diameter, and
wherein the second diameter is greater than the first diameter.

16. The system of claim 15, wherein the first aperture, the second aperture, and the third aperture are positioned adjacent a distal end of the catheter.

17. The system of claim 13, wherein the catheter further defines a fourth aperture, a fifth aperture, a sixth aperture, a seventh aperture, and an eighth aperture,
wherein the first aperture, the second aperture, the third aperture, the fourth aperture, the fifth aperture, the sixth aperture, the seventh aperture, and the eighth aperture are spaced equidistantly along the longitudinal axis,
wherein diameters of the apertures increase from the first aperture positioned proximally, to the eight aperture positioned distally, and
wherein the system is used to deliver a drug along a spine to treat Amyotrophic Lateral Sclerosis (ALS).

18. A computer implemented method of delivering a homogenous fluid, comprising:
determining a plurality of orifice sizes to maintain homogenous flow rates through a catheter; and
controlling a valve of the catheter so that fluid flows through orifices corresponding to the plurality of orifice sizes that will maintain the homogenous flow rates through the catheter.

19. The computer implemented method of claim 18, further comprising controlling a plurality of valves of the catheter to select orifices of the catheter corresponding to the plurality of orifice sizes that will maintain homogeneous flow rates through the catheter.

20. A non-transitory computer readable medium having instructions stored thereon, wherein execution of the instructions by a processor causes the processor to:
receive accelerometer information;
determine a supine position or an upright position based on the accelerometer information;
control operation of a pump based on the determination of the supine position or the upright position; and
provide flow from the pump to a catheter defining a first aperture, a second aperture, and a third aperture,
wherein the first aperture, the second aperture, and the third aperture are spaced equidistantly along a longitudinal axis,
wherein the first aperture defines a first diameter, the second aperture defines a second diameter, and the third aperture defines a third diameter,
wherein the third diameter is greater than the second diameter, and
wherein the second diameter is greater than the first diameter.
